(19) United States
(12) Patent Application Publication
(10) Pub. No.: US 2009/0182407 A1
(43) Pub. Date: Jul. 16, 2009

(54) LUER OR CLAMP-TYPE SUTURE RELEASE APPARATUS AND METHOD FOR LOADING AND DELIVERING A STENT

(75) Inventors: Gary J. Leanna, Holden, MA (US); Mark D. Wood, Shrewsbury, MA (US)

Correspondence Address: HOFFMANN & BARON, LLP, 6900 JERICHO TURNPIKE, SYOSSET, NY, 11791, US

(73) Assignee: Boston Scientific Scimed, Inc., Maple Grove, MN (US)

(21) Appl. No.: 12/352,809

(22) Filed: Jan. 13, 2009

Related U.S. Application Data
(60) Provisional application No. 61/020,822, filed on Jan. 14, 2008.

(51) Int. Cl. A61F 2/06; A61B 19/00

(52) U.S. Cl. 623/1.11; 623/1.12; 128/898

(57) ABSTRACT

An assembly for delivering a self-expanding stent within a body lumen is includes a self-expanding stent, a delivery catheter for delivering the stent, a transfer member removeably engagable with a distal end of the delivery catheter, and an elongate filament for manipulating the stent through the passage of the transfer member. The delivery catheter includes an elongate flexible shaft having a stent holder at its distal end for restraining axial movement of the stent when the stent is disposed within a hollow cylindrical passage of an elongate tube moveably disposed over a portion of the shaft, and a handle secured to the proximal end of the flexible shaft. The elongate filament is releasably secured to the stent, and the ends of the filament are accessible at the handle to permit movement of the stent within the device by manipulation of the filament.

### CROSS-REFERENCE TO RELATED APPLICATIONS

This application claims the benefit of U.S. Provisional Application No. 61/020,822, filed Jan. 14, 2008, the contents of which are incorporated herein by reference.

### FIELD OF THE INVENTION

The present invention relates to a method and system for transporting, loading and delivering a stent, as well as to stent delivery assemblies. More particularly, this invention relates to methods and systems for transferring a stent from a radially expanded state to a radially compressed state prior to surgical implantation.

### BACKGROUND OF THE INVENTION

An intraluminary prosthesis, for example a stent, is a medical device used in the treatment of diseased bodily lumens. A stent is generally a longitudinal tubular device formed of biocompatible material which is useful to open and support various lumens in the body. For example, stents may be used in the bodily vessel, such as in the coronary or peripheral vasculature, esophagus, trachea, bronchi colon, biliary tract, urinary tract, prostate, brain, as well as in a variety of other applications in the body.

A stent generally includes an open flexible configuration which allows the stent to be configured in a radially compressed state for intraluminary catheter implantation. Once properly positioned adjacent the damaged vessel, the stent is radially expanded so as to support and reinforce the vessel. Radial expansion of the stent may be accomplished by inflation of a balloon attached to the catheter or the stent may be of the self-expanding variety which will radially expand once deployed.

Although stent delivery systems are well-known in the art, the assembly of such delivery systems is often complicated. Additionally, contemporary endoscopy practitioners increasingly use polymeric or plastic self-expanding stents. Unlike most metallic self-expanding stents, the polymeric stents have a tendency to permanently deform or lose some of their ability to self-expand when stored in a compressed state for a prolonged period of time. Such stents are therefore generally loaded into the stent delivery system shortly before being implanted in a patient. Such loading, however, often involves numerous steps, requires the use of multiple components (e.g., tools and fixtures) that are not part of the stent delivery system and/or required the practitioner to finish the loading process by manually pushing the stent into the delivery system by hand. Loading a stent in this way is therefore often difficult, time-consuming and has the potential to damage the stent.

Accordingly, there is a need for simplified methods of on-site loading of a stent into stent delivery systems, while minimizing the risk of damaging the stent in the process.

### SUMMARY OF THE INVENTION

The present invention is directed to a method and system for delivering a self-expanding stent into a body lumen. In particular, the present invention relates to an assembly and a method for protecting, loading and delivering a stent in combination with a stent delivery catheter, as well as to overall stent delivery systems.

In one aspect of the present invention is directed to an apparatus for delivering a self-expanding stent within a body lumen. The apparatus may comprise (a) a delivery catheter for delivering the stent, the delivery catheter comprising (i) an elongate flexible shaft; (ii) an elongate tube adapted to enter the body lumen, the tube moveably disposed over a portion of the shaft, the elongate tube comprising a hollow cylindrical passage for containing the stent in its radially compressed state; (iii) a handle secured to the proximal end of the flexible shaft, the handle having at least one detent; (b) a transfer member removeably engagable with a distal end of the delivery catheter, the transfer member comprising a funnel-shaped passage for compressing a stent from an at least partially radially expanded state to an at least partially radially compressed state when the stent is moved through the passage of the transfer member and into the hollow cylindrical passage of the delivery catheter; and (c) an elongate filament for manipulating the stent through the passage of the transfer member, the elongate filament having two opposed ends and a medial portion disposed between the two opposed ends, wherein the elongate filament passes through a handle passage in at least a portion of the handle and further wherein the medial portion is releasably secured to a proximal end of the stent and the two opposed filament ends are accessible at the at least one detent of the handle. The elongate flexible shaft may comprise a stent holder at its distal end for restraining axial movement of the stent when the stent is in at least partially radially compressed state. The at least on detent may comprise at least one open port and at least one enclosure releasably disposed thereat. Desirably, the enclosure comprises at least one cap releasably disposed over the port to cover the port. The elongate filament may comprise a suture, a thread, a cord, a wire and combinations thereof. The elongate filament may pass through a supplemental passage in at least a portion of the flexible shaft. Desirably, at least one end of the filament is releasably secured to the handle of the delivery apparatus. One end of the filament may be releasably secured to the port of the handle and/or may be releasably secured to the cap of the handle. Further, both ends of the filament may be releasably secured to the cap of the handle.

In another aspect of the present invention, an assembly for delivering a self-expanding stent within a body lumen is provided. The assembly may comprise (a) a self-expanding stent; (b) a delivery catheter for delivering the stent, the delivery catheter comprising (i) an elongate flexible shaft; (ii) an elongate tube adapted to enter the body lumen, the tube moveably disposed over a portion of the shaft, the elongate tube comprising a hollow cylindrical passage for containing the stent in its radially compressed state; (iii) a handle secured to the proximal end of the flexible shaft, the handle having at least one detent; (c) a transfer member removeably engagable with a distal end of the delivery catheter, the transfer member comprising a funnel-shaped passage for compressing a stent from an at least partially radially expanded state to an at least partially radially compressed state when the stent is moved through the passage of the transfer member and into the hollow cylindrical passage of the delivery catheter; and (d) an elongate filament for manipulating the stent through the passage of the transfer member, the elongate filament having two opposed ends and a medial portion disposed between the two opposed ends, wherein the elongate filament passes through a handle passage in at least a portion of the handle and further wherein the medial portion is releasably secured to a proximal end of the stent and the two opposed filament ends are accessible at the at least one detent of the handle. The elongate flexible shaft may comprise a stent holder at its distal end for restraining axial movement of the stent when the stent is in at least partially radially compressed state. The at least on detent may comprise at least one open port and at least one enclosure releasably disposed thereat. The enclosure may comprise at least one cap releasably disposed over the port to cover the port. Desirably, the two opposed filament ends are accessible at the port of the handle when the cap is removed from the port. The elongate filament may comprise a suture, a thread, a cord, a wire and combinations thereof. One end or both ends of the filament may be releasably secured to the port of the handle and/or to the cap of the handle. Desirably, the stent is a braided stent, which may comprise a biocompatible material, including but limited to, a biocompatible polymeric material, for example, in some cases, a bioabsorbable or biodegradable polymeric material.

In yet another nonlimiting aspect of the present invention, a method for loading a self-expanding stent into a stent delivery system is provided. The method may comprise (a) providing a self-expanding stent; (b) providing a delivery catheter for delivering the stent, the delivery catheter comprising (i) an elongate flexible shaft; (ii) an elongate tube adapted to enter the body lumen, the tube moveably disposed over a portion of the shaft, the elongate tube comprising a hollow cylindrical passage for containing the stent in its radially compressed state; (iii) a handle secured to the proximal end of the flexible shaft, the handle having at least one detent; (c) providing a transfer member removeably engagable with a distal end of the delivery catheter, the transfer member comprising a funnel-shaped passage and a cylindrical passage, wherein the stent is disposed within the cylindrical passage of the transfer member in an at least partially radially expanded state; (d) providing an elongate filament for manipulating the stent through the passage of the transfer member, the elongate filament having two opposed ends and a medial portion disposed between the two opposed ends, wherein the elongate filament passes through a handle passage in at least a portion of the handle and further wherein the medial portion is releasably secured to a proximal end of the stent and the two opposed filament ends are accessible at the detent of the handle; (e) compressing the stent from the at least partially radially expanded state to an at least partially radially compressed state by moving the stent through the funnel-shaped passage of the transfer member; and (f) moving the stent into the hollow cylindrical passage of the delivery catheter. The elongate flexible shaft may comprise a stent holder at its distal end for restraining axial movement of the stent when the stent is in at least partially radially compressed state. The at least on detent may comprise at least one open port and at least one enclosure releasably disposed thereat. The enclosure may comprise at least one cap releasably disposed over the port to cover the port. Desirably, the two opposed filament ends are accessible at the port of the handle when the cap is removed from the port. Compressing and moving the stent may further comprise moving the elongate filament distally relative to the transfer member. Further, the elongate filament may be removed from the stent after any convenience period, for example after the stent is disposed within the hollow cylindrical passage of the delivery catheter or after the stent is moved outside of the delivery catheter, which is typically done after the transfer member is first removed from the distal end of the delivery catheter. The method may further comprise a step of repositioning the stent, which comprising advancing the elongate flexible shaft distally relative to the elongate tube to move a portion of the stent outside of the hollow cylindrical passage of the delivery catheter; and manipulating the elongate filament proximately in a direction toward the handle to reposition at least part of the portion of the stent back into the hollow cylindrical passage of the delivery catheter.

These and other objectives, features, and advantages of this invention will become apparent from the following detailed description of illustrative embodiments thereof, which is to be read in connection with the accompanying drawings.

### BRIEF DESCRIPTION OF THE DRAWINGS

FIG. 1 illustrates a perspective view of an embodiment of a stent transfer and delivery system in accordance with the subject invention.

FIG. 2 illustrates a plan view of an embodiment of a stent transfer member in cross-section in accordance with the subject invention.

FIG. 3 illustrates an enlarged plan view of a distal portion of the assembly shown in FIG. 1, in cross-section.

FIG. 4 illustrates a plan view of the stent of FIG. 3 further detailing suture loops and suture threads.

FIG. 5 illustrates in a cross-sectional view the stent of FIG. 4 including a covering or a graft.

FIG. 6 illustrates an enlarged plan view of a distal portion of the assembly shown in FIG. 1, after the stent has been loaded in accordance with the subject invention.

FIG. 7 illustrates an enlarged plan view of an embodiment of a distal portion of the distal subassembly in cross-section, in accordance with the subject invention.

FIGS. 8 and 9 illustrate perspective, left side, plan and right side views, respectively, of an embodiment of the proximal handle in accordance with the subject invention.

FIGS. 10 and 11 further illustrate the cap of proximal handle of FIGS. 8 and 9

### DETAILED DESCRIPTION OF THE INVENTION

The present invention relates to an assembly and method for transporting and deploying a stent, or other intraluminary member as described herein, in a bodily passageway. The assembly is suited for medical applications (particularly, endoscopic therapy) in the gastrointestinal tract, the biliary tract, the urinary tract, and the respiratory tract. In particular, a preferred embodiment of the present invention is directed to an assembly and method for transporting, loading and delivering a self-expanding esophageal stent. The system allows the clinician or user to easily load a stent into a delivery system with minimal effort and without damaging the stent. Further, the assembly in accordance with the present invention could also be used in the neurological system (e.g., in the brain) and in the cardiovascular system (e.g., in the heart). Reference to bodily passageways may be to passageways in any of the aforementioned tracts and systems or elsewhere in the body.

It should be noted that references herein to the term “distal” are to a direction away from an operator of the subject invention, while references to the term “proximal” are to a direction towards the operator of the subject invention. Accordingly, when the terms “distal” and “proximal” are used herein in the context of an assembly device that is being deployed within a body, such as a human body, by an operator, the term “distal” refers to a location within the body that is further within the body than a location that is “proximal” to the operator.

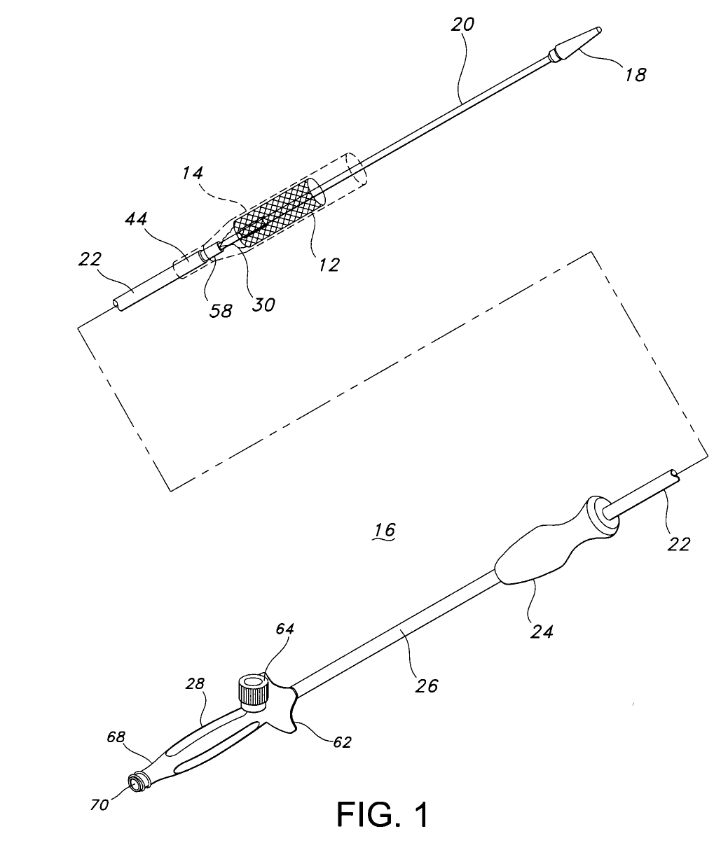

With reference to the drawings, FIG. 1 shows a perspective view of the stent delivery system 10 in accordance with a preferred embodiment of the subject invention. As seen in FIG. 1, a stent 12 is loaded within a stent transfer member 14 which is preferably attached to a stent delivery catheter subassembly 16. The stent delivery catheter subassembly 16 preferably comprises a distal tip 18, a distal inner member 20, an outer tubular member 22, a distal handle 24, a proximal inner member 26, and a proximal handle 28, interrelated as shown. Further, as described in detail below, the stent delivery catheter subassembly 16 further includes a loading suture 30, which is removeably coupled to the stent 12 and extends through the stent delivery catheter subassembly 16 to the proximal handle 28.

While the present invention can be applied to the delivery of many intraluminary devices, it is particularly suited for delivering a self-expanding stent 12. A preferred stent 12 should be capable of being radially compressed and longitudinally extended for implantation into a bodily lumen. The degree of elongation depends upon the structure and materials of the stent, and may be quite varied. The diameter of the stent also may become several times smaller as it elongates. It is preferred that the stent 12 be constructed to self-expand when released from a radially compressed state. Any stent that is capable of radial expansion is preferably used in accordance with the present invention. Further, the stent 12 may be repositionable, removable and/or reconstrainable, and/or may include multiple interconnected or non-interconnected stents. Various stent types and stent constructions may be employed in the invention, and the invention can be constructed to accommodate stents of various sizes and configurations.

One embodiment applies the method and system of the present invention to a braided stent 12. As used herein the term braiding and its variants refer to the diagonal intersection of elongate filaments, such as elongate wires, so that each filament passes alternately over and under one or more of the other filaments, which is commonly referred to as an intersection repeat pattern. Useful braiding patterns include, but are not limited to, a diamond braid having a 1/1 intersection repeat pattern, a regular braid having a 2/2 intersection repeat pattern or a hercules braid having a 3/3 intersection repeat pattern. The passing of the filaments under and over one and the other results in slidable filament crossings that are not interlooped or otherwise mechanically engaged or constrained.

While the stent 12 can be formed of metals, plastics or other materials, it is preferred that a biocompatible construction is employed. Useful biocompatible materials include but are not limited to biocompatible metals, biocompatible alloys, biocompatible polymeric materials, including synthetic biocompatible polymeric materials and bioabsorbable or biodegradable polymeric materials, materials made from or derived from natural sources and combinations thereof. Useful biocompatible metals or alloys include, but not limited to, nitinol, stainless steel, cobalt-based alloy such as Elgiloy, platinum, gold, titanium, tantalum, niobium, polymeric materials and combinations thereof. Useful synthetic biocompatible polymeric materials include, but are not limited to, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, silks and polytetrafluoroethylenes. The polymeric materials may further include a metallic, a glass, ceramic or carbon constituent or fiber. Useful and nonlimiting examples of bioabsorbable or biodegradable polymeric materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like. Further, the stent 12 may include materials made from or derived from natural sources, such as, but not limited to collagen, elastin, glycosaminoglycan, fibronectin and laminin, keratin, alginate, combinations thereof and the like.

Further, the stent 12 may be made from polymeric materials which may also include radiopaque materials, such as metallic-based powders or ceramic-based powders, particulates or pastes which may be incorporated into the polymeric material. For example, the radiopaque material may be blended with the polymer composition from which the polymeric filament is formed, and subsequently fashioned into the stent as described herein. Alternatively, the radiopaque material may be applied to the surface of the metal or polymer stent. Various radiopaque materials and their salts and derivatives may be used including, without limitation, bismuth, barium and its salts such as barium sulfate, tantalum, tungsten, gold, platinum and titanium, to name a few. Additional useful radiopaque materials may be found in U.S. Pat. No. 6,626,936, which is herein incorporated in its entirely by reference. Metallic complexes useful as radiopaque materials are also contemplated. The stent 12 may be selectively made radiopaque at desired areas along the stent 12 or made be fully radiopaque, depending on the desired end-product and application. Further, portions of the stent 12, for example stent filaments, may have an inner core of tantalum, gold, platinum, iridium or combination of thereof and an outer member or layer of nitinol to provide a composite filament for improved radiocapicity or visibility. Alternatively, the stent 12 may also have improved external imaging under magnetic resonance imaging (MRI) and/or ultrasonic visualization techniques. MRI is produced by complex interactions of magnetic and radio frequency fields. Materials for enhancing MRI visibility include, but are not limited to, metal particles of gadolinium, iron, cobalt, nickel, dysprosium, dysprosium oxide, platinum, palladium, cobalt based alloys, iron based alloys, stainless steels, or other paramagnetic or ferromagnetic metals, gadolinium salts, gadolinium complexes, gadopentetate dimeglumine, compounds of copper, nickel, manganese, chromium, dysprosium and gadolinium. To enhance the visibility under ultrasonic visualization the stent 12 of the present invention may include ultrasound resonant material, such as but not limited to gold. Other features, which may be included with the stent 12 of the present invention, include radiopaque markers; surface modification for ultrasound, cell growth or therapeutic agent delivery; varying stiffness of the stent or stent components; varying geometry, such as tapering, flaring, bifurcation and the like; varying material; varying geometry of stent components, for example tapered stent filaments; and the like.

The stent transfer member 14 is preferably intended to protect a stent 12 or other similar inter-luminary device, before and during the time it is loaded into a delivery catheter lumen. Also, the stent transfer member 14 serves to safely radially compress the stent 12 for loading into a catheter lumen. In this way, the stent 12 can be loaded into the catheter lumen just prior to implantation in a patient's bodily passageway.

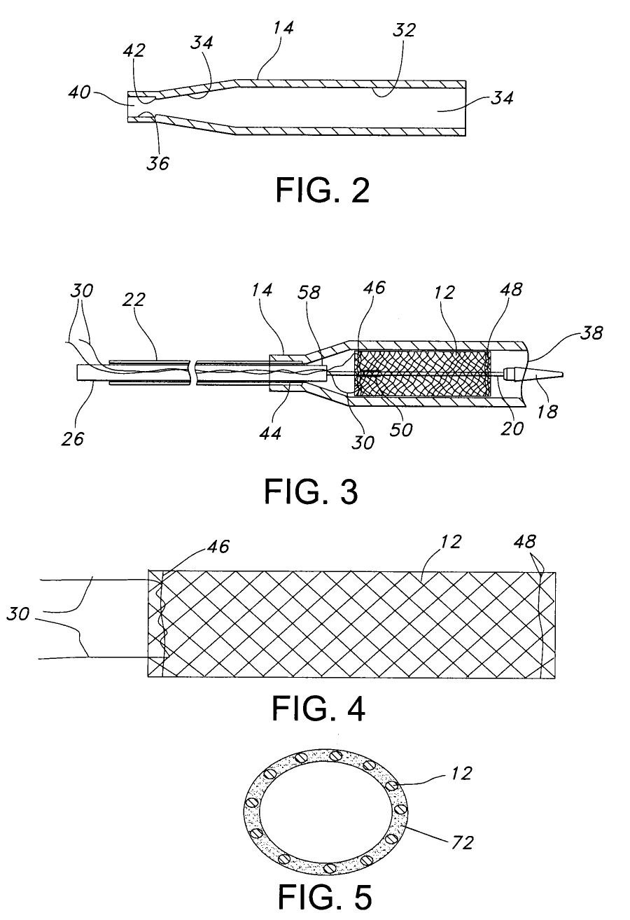

As shown in FIG. 2, an embodiment of the stent transfer member 14 is seen in cross-section separated from the overall delivery system 10. The stent transfer member 14 preferably has a stent holding passage 32 whose inner diameter is preferably adapted to enclose a self-expanding stent in a fully radially expanded state. Alternatively, the stent holding passage 32 could have a somewhat smaller inner circumference in order to provide an element of frictional engagement with a stent 12 loaded therein. Further, although the stent holding passage 32 preferably encloses the entire length of the stent 12, it could be longer or shorter. Thus, the holding passage 32 could be made to encircle only a portion of the stent 12. The stent transfer member 14 also preferably includes a compression funnel passage 34 which serves to radially compress a stent 12 that passes from the stent holding passage to the more proximal catheter receiving passage 36. The distal end 38 of the stent transfer member 14 is preferably open to allow unobstructed passage of the distal portions of the stent delivery catheter subassembly 16. The proximal end 40 of the stent transfer member 14 is preferably adapted to engage with a distal end 44 of the outer tubular member 22. Desirably, the proximal end 40 of the stent transfer member 14 has an inner cylindrical portion which acts as a catheter receiving passage 36 having a circumference that engages the outer circumference of the distal end 44 of the outer tubular member 22. A transition step 42 preferably serves as a mating seat for the outer tubular member 22. The transition step 42 is desirable to radially compress the stent 12 to the same or similar diameter as the inner lumen of the outer tubular member 22.

The length or diameter of the stent transfer member 14 may be constructed to suit a particular application and/or stent. Also, the edges of the stent transfer member 14 could have a beveled profile. Further, the transfer member 14 could be constructed with one or more longitudinal slits or slots that can extend along the entire length or only a portion of the transfer member 14. As a further alternative, the transfer member 14 could engage the outer tubular member 22 using other coupling techniques. Further still, as discussed above with regard to stents 12, the transfer member 14 could be coated. Such coatings could reduce or enhance frictional engagement. Additionally, such coatings could further be designed to transfer or adhere to the stent 12 after it is removed from the transfer member 14.

While the stent transfer member 14 is shown as a unitary member, it can alternatively be formed by separate elements or separable elements. In such a manner, the stent transfer member 14 could be made to split open or have a portion that can be removed to facilitate loading the stent 12 therein. For example, the stent transfer member 14 could be fractured, slit, split or hinged to provide the separate or separable elements. Such fractures, slits, splits, hinges, sites or detents for providing the separability function, either in part or in total, may be disposed along a longitudinal portion or axis of the stent transfer member 14, along a circumferential portion or axis of the stent transfer member 14 and/or a combination thereof. Such an embodiment would, however, preferably provide some mechanism for holding the separate or separable elements together. Also, although a generally cylindrical outer structure is illustrated in FIG. 2, the stent transfer member 14 could have almost any shape to its outer surfaces. Whether to provide ergonomic features, a handle, engagement surfaces for tools, or simply ease of manufacture, it should be understood that the outer surfaces of the stent transfer member 14 could be altered from that shown. With regard to the inner surfaces 32, 34, 36 a cylindrical configuration is preferred, but alternative shapes are anticipated.

With reference to FIGS. 3 and 4, in accordance with the present invention a loading suture 30 or other suitable thread is preferably interlaced, braided, threaded, woven or otherwise disposed into a proximal end the stent 12. The loading suture 30, desirably a central or medial portion thereof, may be interlaced, braided, threaded, woven or otherwise disposed directly into or onto the wires, filaments or structure of the stent 12 itself. The central or medial portion of the loading suture 30 may be interlaced, braided, threaded, woven or otherwise disposed directly onto separate retrieval suture 46 that is part of the stent 12. The loading suture 30 can be threaded through any number of loops of the proximal retrieval suture 46. Further, the central or medial portion of the loading suture 30 may engage the wires, filaments or structure of the stent 12 and/or engage the retrieval suture 46. The two ends of the loading suture 30 then preferably extend proximally from the stent 12 to the proximal end of the delivery system 10.

Further, as depicted in FIGS. 3 and 4, retrieval sutures 48, 46 may located at either or both the distal and proximal ends of the stent 12. The present invention, however, is not so limited, and the retrieval sutures 48, 46 may located at any point or points along the longitudinal and/or circumferential axis, individually or in combination, of the stent 12. The retrieval sutures 48, 46 may be useful to a physician or practitioner after the stent is delivered into a body lumen. Such sutures 48, 46 desirably remain on the stent after it is implanted and allow the practitioner to reposition and/or remove the stent. Devices such as graspers or hooks can be used to pull on the retrieval sutures 48, 46. When pulled, the retrieval suture 48, 46 is preferably adapted to constrict or radially contract the end of the stent in a purse string type movement. This constriction of an end of the stent 12 further makes it easier for the stent 12 to be pulled through an intraluminary passage of the stent transfer member 14 and into the stent delivery catheter subassembly 16.

It should be noted that references herein to the term “suture” denotes a length of thread, thread-like member, cord, filament, wire or other similar structure. It should be understood that sutures as referred to herein can be made of a single material or composite materials. Accordingly, the terms “suture,” “thread,” “cord,” “filament,” and/or “wire” are used interchangeably herein.

As seen in FIG. 3, once the loading suture 30 is coupled to the stent 12 and fed through the delivery catheter subassembly 16 toward the proximal end of the assembly, the stent 12 is preferably loaded into the stent holding passage 32. This can be done before or after the stent transfer member is mounted onto the distal end 44 of the outer tubular member 22. The configuration shown in FIG. 3 maintains the stent 12 in a radially expanded state and can serve to protect the stent from the time of assembly until the stent is loaded into the lumen of outer tubular member 22. Thus, the stent need not be compressed into a delivery catheter for an extended period, potentially causing permanent deformation.

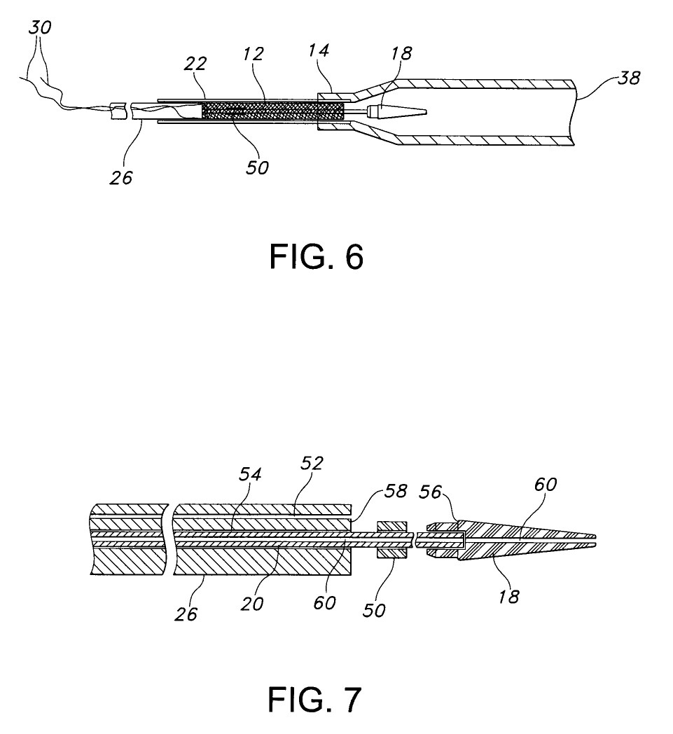

With reference to FIGS. 3 and 6, the distal inner member 20 and the proximal inner member 26 are preferably fixed to one another, functioning as a unitary member, along with distal tip 18. These three inner members 18, 20, 26 are preferably coaxially configured within outer tubular member 22. Also, as with a more traditional delivery catheter, the outer tubular member 22 is slidable axially relative to the three inner members 18, 20, 26. Further, when the stent transfer member 14 is mounted onto the distal end 44, it preferably slides axially in conjunction with the outer tubular member 22, and thus also relative to inner members 18, 20, 26. Thus, two handles are provided for manually sliding these elements relative to one another. The distal handle 24 controls the sliding movement of the outer tubular member 22, along with, if attached, stent transfer member 14. The proximal handle 28 likewise controls the sliding movement of the above mentioned inner members 18, 20, 26. This relative sliding movement is used for both loading and deployment (delivery) of the stent 12.

The inner members 20 and 26 and outer member 22 are preferably formed of a body compatible material. Desirably, the biocompatible material is a biocompatible polymer. Examples of suitable biocompatible polymers include, but are not limited to, polypropylene (PP), polytetrafluoroethylene (PTFE), polyethylene terephthalate (PET), high density polyethylene (HDPE) and the like. Materials for the members 20, 22, 26 may be the same or different. Additionally, the outer member 22 and the stent transfer member 14 could have coverings, films, coatings, and the like, desirably a polymeric covering, disposed over the inner surfaces to aid in the loading and/or deployment of the stent 12.

The loading suture 30 preferably provides a link or coupling means between the stent 12 and the proximal end of the proximal inner member 26. In a preferred embodiment, the loading suture 30 is removably secured to the proximal handle 28. The desired purpose of securing the loading suture 30 is to limit the relative axial movement of the stent 12 away from the proximal inner member 26 and/or the proximal handle 28. Thus, by moving the distal handle 24 away from the proximal handle 28, the stent 12 is caused to be drawn through the compression funnel passage 34 and into an inner lumen of the outer tubular member 22, as seen in FIG. 6. During this movement, the stent 12 is transferred from the stent holding passage 32 to the inner lumen of the outer tubular member 22. Also, during this movement the stent 12 is preferably made to radially compress and engage onto the distal inner member 20, or at least engage with the stent holder 50. Thus, the configuration seen in FIG. 6 shows the stent 12 fully loaded within the inner lumen of the outer tubular member 22. Alternatively, the distal end 44 of the outer tubular member 22 can include an inner bevel to aid in loading the stent 12.

In one embodiment, the loading suture 30 can simply be threaded between the outer surface of the proximal inner member 26 and the inner surface of the outer tubular member 22. Alternatively, the loading suture can be made to pass through auxiliary passage 52 in the proximal inner member 26, seen in FIG. 7. The loading suture 30 can also be made to pass through the entire length of the proximal inner member 26, the inside of the proximal handle 28 and out the proximal end of the proximal handle 28. Alternatively, an additional opening can be made in the outer surface of the proximal inner member 26, allowing the loading suture 30 to exit the auxiliary passage 52. Such a loading suture 30 exit (not shown) can be disposed on the proximal inner member 26 at a location between the distal handle 24 and proximal handle 28. Further, this alternative suture opening in the proximal inner member 26 can correspond with a stent release position discussed below.

During deployment or delivery of the stent 12, as the two handles 24, 28 are drawn toward one another, there is a particular distance between them that corresponds with release of the stent 12. As the proximal edge of the distal handle 24 slides along the surface of the proximal inner member 26, a position on the surface of the proximal inner member 26 will correspond with the position that releases the stent 12 from the outer tubular member 22. Thus, a marker for this release position can be provided on the surface of the proximal inner member 26. Alternatively, the loading suture opening could be positioned to also function as this type of marker.

Also as depicted in FIG. 5, the materials of the stent 12 as well as the component filaments of the stent 12 can be further enhanced with coverings, films, coatings, and other materials and techniques. A covering, films or coating 72 may be in the form of a tubular structure, for example composed of polymeric material and/or silicone. The covering may also comprise any plastic or polymeric material, desirably a somewhat hard but flexible plastic or polymeric material. The covering may be transparent or translucent, desirably substantially or partially transparent. Furthermore, the covering may be constructed of any suitable biocompatible materials, such as, but not limited to, polymers and polymeric materials, including fillers such as metals, carbon fibers, glass fibers or ceramics. Useful covering materials include, but are not limited, polyethylene, polypropylene, polyvinyl chloride, polytetrafluoroethylene, including expanded polytetrafluoroethylene (ePTFE), fluorinated ethylene propylene, fluorinated ethylene propylene, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polyimides, polycarbonates, polyaldehydes, polyether ether ketone, natural rubbers, polyester copolymers, styrene-butadiene copolymers, polyethers, such as fully or partially halogenated polyethers, and copolymers and combinations thereof. The coating or coatings 72 may be on the stent 12, components of the stent 12, and combinations thereof. The stent components, in part or in total, may be temporary, for example bioabsorbable, biodegradable, and the like, or may be permanent (i.e., not substantially bioabsorbable or biodegradable), for example the above-described biocompatible metals, alloys and polymers.

Further, the stent 12 and/or covering 72 may be treated with any of the following: anti-thrombogenic agents (such as heparin, heparin derivatives, urokinase, and PPack (dextrophenylalanine proline arginine chloromethylketone); anti-proliferative agents (such as enoxaprin, angiopeptin, or monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-miotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides); vascular cell growth promotors (such as growth factor inhibitors, growth factor receptor antagonists, transcriptional activators, and translational promotors); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

With reference to FIG. 7, the distal inner member 20 is preferably disposed and secured within an inner passage 54 in the proximal inner member 26. Similarly, the stent holder 50 and the distal tip 18 are preferably fixed to the distal inner member 20. These elements can be secured by frictional engagement or more permanent bonding. Alternatively, the distal inner member 20 and the proximal inner member 26 could be made as one unitary member. In this way, the distal subassembly seen in FIG. 7 will preferably move in unison. Also, the distal inner member 20 preferably extends through the entire length of the proximal inner member 26 and beyond it in the distal direction.

The distance between the distal end 58 of the proximal inner member 26 and the proximal end 56 of the distal tip 18 is preferably suited to accommodate the stent 12 in a radially compressed state, as seen in FIG. 6. It should be noted that as the stent 12 is radially compressed, it tends to axially expand. Thus, it is desired that the distance between the proximal inner member 26 and the distal tip 18 comfortably accommodate the axially expanded state of the stent 12. The stent holder 50 is preferably provided to enhance the frictional engagement between the stent 12 and the distal inner member 20. Once compressed onto the stent holder 50, the stent 12 will slide axially in unison with the distal inner member 20, unless the stent is radially released. Thus, as seen in FIG. 6, once compressed into the outer tubular member 22, at least a portion of the stent 12 is preferably engaged with the stent holder 50. Preferably, the stent holder 50 is made of a soft deformable or low durometer polymer that allows it to conform to the inner surface of the stent. For example, the stent holder 50 could be made from 2533 Pebax® (ARKEMA, Courbevoie, France) a hardness 25, shore D, non-plasticized flexible polyamide, silicone, thermoplastic elastomer, such as but not limited to Dynaflex® (GLS Corp., McHenry, Ill.), thermoplastic polyurethane elastomer and any other suitable material. However, it is understood that other suitable materials that function to enhance engagement with the stent could be used. Additionally, although the stent holder 50 is shown as an annular band, alternatively it could extend around only a portion or separate portions of the distal inner member 20. In other words, the stent holder 50 may not have to completely encompass the distal inner member 20, but may be only partially disposed around a circumferential portion thereof. Moreover, the stent holder 50 may have a pattern, such as a surface pattern of indentations and/or protrusions for facilitating securement of the stent 12. In some embodiments, the stent holder 50 may have barbs, pins or protrusions which may engage the stent 12. Further, with any of the embodiments, the device or system may include multiple stent holders 50, either axially spaced apart or axially juxtaposed. The stent holder 50 may be short, i.e., having a longitudinal dimension encompassing only a portion of the longitudinal expanse of the stent 12, or may be long, i.e., having a longitudinal expanse encompassing a substantial portion of the longitudinal expanse of the stent 12.

Once the stent 12 is loaded as seen in FIG. 6, the stent transfer member 14 and/or the loading suture 30 can be removed from the assembly. Thereafter, the delivery catheter subassembly 16 that remains is preferably used to deliver the stent 12, into a body lumen. In one alternative embodiment, the loading suture 30 need not be removed before delivery of the stent 12 into the patient. In this alternative embodiment, the loading suture 30 can be used to adjust the axial delivery position of the stent 12 in the proximal direction within the body lumen. In other words, the loading suture 30 is thus used to pull the stent 12 back to a more proximal location. Once positioned as desired, the loading suture 30 can be removed from the stent 12 as discussed below.

Removal of the stent transfer member 14 is preferably relatively simple. A frictional mounting between the applicable two elements 14, 44 is desirable, for easy removal. Alternatively, a screw-thread or other known means of engagement between the two elements could be provided.

With regard to the removal of the loading suture 30, at least one end the loading suture 30 is preferably detached from the proximal handle 28 or the proximal inner member 26, where it was secured. Then, by detaching and pulling the other end of the loading suture 30, it is preferably pulled out of the stent 12 and the overall assembly. The loose weaving or braiding configuration between the loading suture 30 and the retrieval suture 46, allows the suture 30 to be removed in this way. Even if the loading suture 30 were woven into the stent 12 itself, this removal technique could still suitably be used. Once the loading suture 30 has been removed from the stent 12 and the stent delivery catheter subassembly 16, it can be set aside and/or discarded. In this way, with the stent transfer member 14 and loading suture 30 removed, the stent delivery catheter subassembly 16 is now loaded with a stent for delivery and can be inserted into a body lumen for delivery. The present invention, however, is not so limited to the above-described technique. For example, the loading suture 30 could be a biodegradable, bioabsorbable or dissolvable. In such as case the suture 30 may be left in vivo.

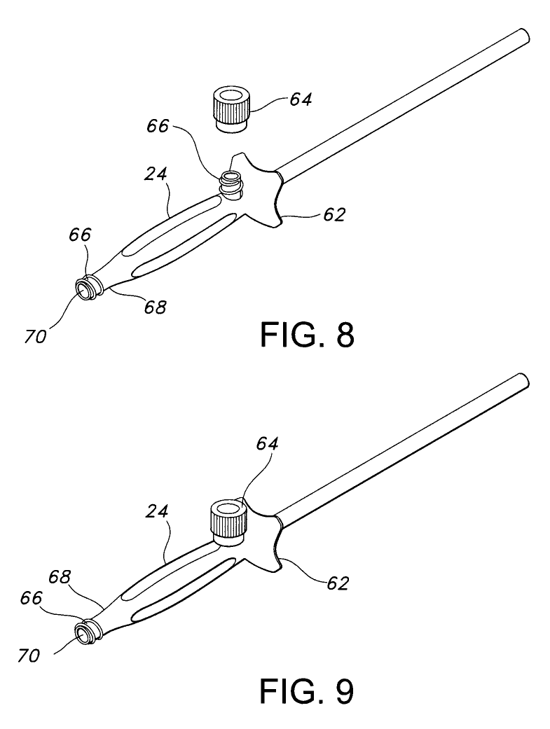

FIGS. 8 and 9 further illustrate the proximal handle 28, which may be also referred to as a luer body. The proximal handle 28 shown in FIGS. 8 and 9 may include an auxiliary passage (not shown) that corresponds to auxiliary passage 52 in the proximal inner member 26. Accordingly, the loading suture 30 may be fed through these auxiliary passages. The auxiliary passage of the proximal handle 28 may end either near or at either of the ports 66, 70. Additionally, a luer flange 62 or other similar ergonomic feature may be provided on the distal end of the proximal handle 28. The proximal handle 28 may further include a screw-on cap 64 as a suture lock. The screw-on cap 64 is preferably adapted to be mounted on a portion of the proximal handle 28, for example onto a threaded cylindrical port 66 protruding from a lateral surface of the handle 28. Alternatively, the same screw threading can be molded onto the proximal end 68 (depicted as port 70) to receive the screw-on cap 64. As a further alternative, the two ends of the loading suture 30 can be wrapped around either threaded cylindrical port 70, 66 before being secured by the cap 64. Additionally, the loading suture 30 can be fed through an inner passage in the port 66 or the flush passage 70, and then secured by the screw-on cap 64. As yet a further alternative, the mounting locations could engage the cap 64 with a snap lock design, rather than screw threads.

As part of an overall stent delivery system, it is preferred that an inner passage be provided for flushing fluids through the stent delivery catheter subassembly 16. In this way, the proximal handle 28 is preferably provided with a flush passage 70 which traverses the length of the proximal handle 28. This flush passage 70 is preferably open to and in communication with inner passage 52 in the distal inner member 20. Further, this inner flow passage should preferably extend all the way through to an inner passage 60 in the distal tip 18. In this way, an inner flush passage is provided from end to end in the overall assembly. Additionally, the proximal end of the proximal handle 28 can be molded to receive fluid flushing attachments or other surgical instruments.

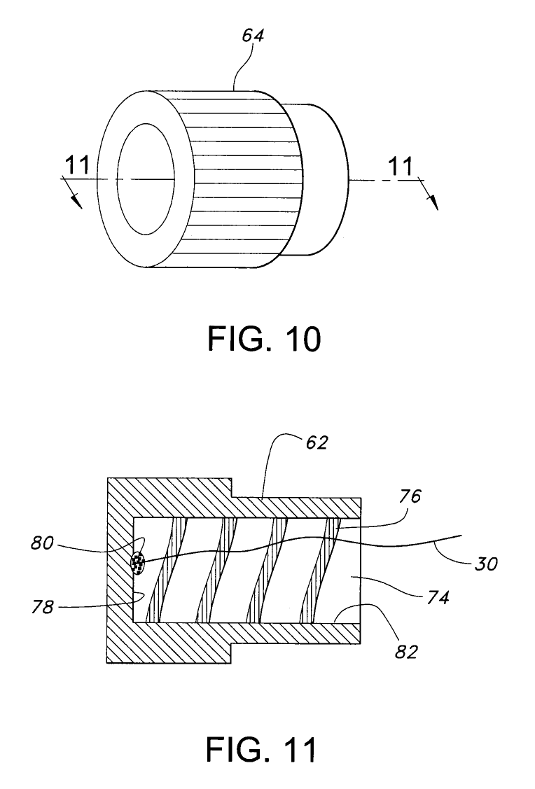

As depicted in FIGS. 9 and 10, one end of the loading suture 30 may be permanently or releasably attached to one of the screw-on cap 64. This can be done by injection molding, adhesives, heated bonding or other techniques. For example, the one end of the suture thread 30 may be releasably or permanently secured by adhesive or bonding agent 80 at an inner top portion 78 of the cap 64. Alternatively, the one end of the suture thread 30 may be releasably or permanently secured at an inner wall portion 82 of the cap 64 defined by the open bore 74 having grooves 76 engagable with the threaded portions cylindrical ports 66, 70. It is desirable to allow the other end to be removable so that it can be pulled out of the stent 12 and the assembly 10, as discussed above. As a further alternative, both ends of the loading suture 30 could be permanently or releasably secured, thus requiring the surgeon or user to simply cut them off prior to pulling the loading suture out. The ports 66 and/or 70 may secure the sutures in any number of manners. For example, the assembly 10 may include any suitable detent, including an enclosure, including but not limited to a cap, for releasably or permanently securing the thread 30 by, for example, a pinching mechanism, securing hooks or projections, tying techniques, and the like, or combinations thereof.

Additional features of useful stent delivery systems are further described in U.S. Patent Application Publication Nos. 2007-02790931 A1, 2007-02790932 A1 and 2007-02790937 A1, the contents of which are incorporated herein by reference in their entirety.

Further, any of the components of the assembly 10 may have specific surface texturing and/or coatings to improve or affect loading, deployment and/or movement of the assembly 10 or its components.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or throughout the specification may be used with one and another without limitation.

What is claimed is:

1. An apparatus for delivering a self-expanding stent within a body lumen comprising:
(a) a delivery catheter for delivering the stent, the delivery catheter comprising:
(i) an elongate flexible shaft;
(ii) an elongate tube adapted to enter the body lumen, the tube moveably disposed over a portion of the shaft, the elongate tube comprising a hollow cylindrical passage for containing the stent in its radially compressed state;
(iii) a handle secured to the proximal end of the flexible shaft, the handle having at least one detent;
(b) a transfer member removeably engagable with a distal end of the delivery catheter, the transfer member comprising a funnel-shaped passage for compressing a stent from an at least partially radially expanded state to an at least partially radially compressed state when the stent is moved through the passage of the transfer member and into the hollow cylindrical passage of the delivery catheter; and
(c) an elongate filament for manipulating the stent through the passage of the transfer member, the elongate filament having two opposed ends and a medial portion disposed between the two opposed ends,
wherein the elongate filament passes through a handle passage in at least a portion of the handle and further wherein the medial portion is releasably secured to a proximal end of the stent and the two opposed filament ends are accessible at the at least one detent of the handle.

2. The apparatus of claim 1, wherein the elongate flexible shaft comprises a stent holder at its distal end for restraining axial movement of the stent when the stent is in at least partially radially compressed state.

3. The apparatus of claim 1, wherein the at least on detent comprises at least one open port and at least one enclosure releasably disposed thereat.

4. The apparatus of claim 3, wherein the enclosure comprises at least one cap releasably disposed over the port to cover the port.

5. The apparatus of claim 4, wherein the two opposed filament ends are accessible at the port of the handle when the cap is removed from the port.

6. The apparatus of claim 1, wherein the elongate filament comprises a suture, a thread, a cord, a wire and combinations thereof.

7. The apparatus of claim 1, wherein the elongate filament passes through a supplemental passage in at least a portion of the flexible shaft.

8. The apparatus of claim 1, wherein at least one end of the filament is releasably secured to the handle.

9. The apparatus of claim 8, wherein the at least one end of the filament is releasably secured to the port of the handle.

10. The apparatus of claim 8, wherein the at least one end of the filament is releasably secured to the cap of the handle.

11. The apparatus of claim 1, wherein the ends of the filament are releasably secured to the cap of the handle.

12. The apparatus of claim 1, wherein the hollow cylindrical passage of the delivery catheter is sized to surround the entire length of the stent.

13. An assembly for delivering a self-expanding stent within a body lumen comprising:
(a) a self-expanding stent;
(b) a delivery catheter for delivering the stent, the delivery catheter comprising:
(i) an elongate flexible shaft;
(ii) an elongate tube adapted to enter the body lumen, the tube moveably disposed over a portion of the shaft, the elongate tube comprising a hollow cylindrical passage for containing the stent in its radially compressed state;
(iii) a handle secured to the proximal end of the flexible shaft, the handle having at least one detent;
(c) a transfer member removeably engagable with a distal end of the delivery catheter, the transfer member comprising a funnel-shaped passage for compressing a stent from an at least partially radially expanded state to an at least partially radially compressed state when the stent is moved through the passage of the transfer member and into the hollow cylindrical passage of the delivery catheter; and
(d) an elongate filament for manipulating the stent through the passage of the transfer member, the elongate filament having two opposed ends and a medial portion disposed between the two opposed ends,
wherein the elongate filament passes through a handle passage in at least a portion of the handle and further wherein the medial portion is releasably secured to a proximal end of the stent and the two opposed filament ends are accessible at the at least one detent of the handle.

14. The assembly of claim 13, wherein the elongate flexible shaft comprises a stent holder at its distal end for restraining axial movement of the stent when the stent is in at least partially radially compressed state.

15. The assembly of claim 13, wherein the at least on detent comprises at least one open port and at least one enclosure releasably disposed thereat.

16. The assembly of claim 15, wherein the enclosure comprises at least one cap releasably disposed over the port to cover the port.

17. The assembly of claim 16, wherein the two opposed filament ends are accessible at the port of the handle when the cap is removed from the port.

18. The assembly of claim 13, wherein the elongate filament comprises a suture, a thread, a cord, a wire and combinations thereof.

19. The assembly of claim 13, wherein the at least one end of the filament is releasably secured to the port of the handle.

20. The assembly of claim 13, wherein the at least one end of the filament is releasably secured to the cap of the handle.

21. The assembly of claim 13, wherein the ends of the filament are releasably secured to the cap of the handle.

22. The assembly of claim 13, wherein the stent is a braided stent.

23. The assembly of claim 13, wherein the stent comprises a biocompatible material.

24. The assembly of claim 13, wherein the stent comprises a biocompatible polymeric material.

25. The assembly of claim 13, wherein the stent comprises a bioabsorbable or biodegradable polymeric material.

26. A method for loading a self-expanding stent into a stent delivery system comprising:
(a) providing a self-expanding stent;
(b) providing a delivery catheter for delivering the stent, the delivery catheter comprising:
(i) an elongate flexible shaft;
(ii) an elongate tube adapted to enter the body lumen, the tube moveably disposed over a portion of the shaft, the elongate tube comprising a hollow cylindrical passage for containing the stent in its radially compressed state;
(iii) a handle secured to the proximal end of the flexible shaft, the handle having at least one detent;
(c) providing a transfer member removeably engagable with a distal end of the delivery catheter, the transfer member comprising a funnel-shaped passage and a cylindrical passage, wherein the stent is disposed within the cylindrical passage of the transfer member in an at least partially radially expanded state;
(d) providing an elongate filament for manipulating the stent through the passage of the transfer member, the elongate filament having two opposed ends and a medial portion disposed between the two opposed ends, wherein the elongate filament passes through a handle passage in at least a portion of the handle and further wherein the medial portion is releasably secured to a proximal end of the stent and the two opposed filament ends are accessible at the detent of the handle;
(e) compressing the stent from the at least partially radially expanded state to an at least partially radially compressed state by moving the stent through the funnel-shaped passage of the transfer member; and
(f) moving the stent into the hollow cylindrical passage of the delivery catheter.

27. The method of claim 26, wherein the elongate flexible shaft comprises a stent holder at its distal end for restraining axial movement of the stent when the stent is in at least partially radially compressed state.

28. The method of claim 26, wherein the at least on detent comprises at least one open port and at least one enclosure releasably disposed thereat.

29. The method of claim 28, wherein the enclosure comprises at least one cap releasably disposed over the port to cover the port.

30. The method of claim 29, wherein the two opposed filament ends are accessible at the port of the handle when the cap is removed from the port.

31. The method of claim 26, wherein the steps of compressing and moving the stent further comprise moving the elongate filament distally relative to the transfer member.

32. The method of claim 31, wherein the steps of compressing and moving the stent further comprise constricting the proximal end of the stent as the elongate filament is moved distally relative to the transfer member.

33. The method of claim 31, wherein the step of moving the elongate filament distally relative to the transfer member further comprises pulling the elongate member in a distal direction.

34. The method of claim 26, further comprising removing elongate filament from the stent.

35. The method of claim 34, wherein the step of removing the elongate filament from the stent is performed after the stent is disposed within the hollow cylindrical passage of the delivery catheter.

36. The method of claim 34, further comprising:
removing the transfer member from the distal end of the delivery catheter; and
moving the stent outside of the delivery catheter.

37. The method of claim 36, wherein the step of removing the elongate filament from the stent is performed after the stent is moved outside of the delivery catheter.

38. The method of claim 26, further comprising a step of repositioning the stent comprising:
advancing the elongate flexible shaft distally relative to the elongate tube to move a portion of the stent outside of the hollow cylindrical passage of the delivery catheter; and
manipulating the elongate filament proximately in a direction toward the handle to reposition at least part of the portion of the stent back into the hollow cylindrical passage of the delivery catheter.
